(19) United States
(12) Patent Application Publication
(10) Pub. No.: US 2023/0081417 A1
(43) Pub. Date: Mar. 16, 2023

(54) RESUSCITATION DEVICE

(71) Applicant: Joltz, LLC, Passaic, NJ (US)

(72) Inventors: Jonathan Rosenblum, Bet Shemesh (IL); Yehuda Roseman, Bet Shemesh (IL); Daniel Masse, Windham, NH (US); Shaun Welches, Townsend, MA (US); Michael Kishinevsky, North Andover, MA (US); Sam McGinn, Billerica, MA (US)

(73) Assignee: Joltz, LLC, Passaic, NJ (US)

(21) Appl. No.: 17/920,042

(22) PCT Filed: Apr. 21, 2021
(86) PCT No.: PCT/IL2021/050453

§ 371 Date: Oct. 20, 2022

Related U.S. Application Data
(60) Provisional application No. 63/014,127, filed on Apr. 23, 2020.

(51) Int. Cl. A61H 31/00; G16H 40/67

(52) U.S. Cl. CPC A61H 31/005; G16H 40/67

(57) ABSTRACT

Disclosed herein is a resuscitation device facilitating the administration of cardiopulmonary resuscitation to a subject, the resuscitation device comprising a housing having a top surface and a bottom surface, said top surface having a concave dell configured to guide on the top surface a hand positioning of a rescuer administrating a cardiopulmonary resuscitation to a subject, and said bottom surface configured to position and stabilize the housing over a sternum of the subject, and wherein the housing is configured to transmit a uniform distribution of the cardiopulmonary resuscitation force to the chest of the subject, said uniform distribution facilitates distributing the cardiopulmonary resuscitation force over a surface area that greater than the area of the top surface that directly receives the cardiopulmonary resuscitation force, thereby facilitating injury and contusion prevention to ribs and the sternum of the subject.

### FIELD OF THE INVENTION

The present disclosure generally relates to resuscitation devices.

### BACKGROUND

A primary task of the heart is to pump oxygenated, nutrient-rich blood throughout the body. Electrical impulses generated by a portion of the heart regulate the pumping cycle. When the electrical impulses follow a regular and consistent pattern, the heart functions normally and the pumping of blood is optimized. When the electrical impulses of the heart are disrupted (i.e., cardiac arrhythmia), sudden cardiac arrest may result, which inhibits the circulation of blood. As a result, the brain and other critical organs are deprived of nutrients and oxygen. A person experiencing sudden cardiac arrest may suddenly lose consciousness and die shortly thereafter if left untreated.

A well-known and effective treatment for sudden cardiac arrest or arrhythmia is cardiopulmonary resuscitation (“CPR”). CPR is an emergency procedure that combines chest compressions with artificial ventilation in an effort to manually preserve intact brain function until further measures are taken to restore spontaneous blood circulation and breathing in a person who is in cardiac arrest. CPR involves chest compressions for adults between 5 centimeters (2.0 inches) and 6 centimeters (2.4 inches) deep and at a rate of at least 100 to 120 per minute. The rescuer may also provide artificial ventilation by either exhaling air into the subject's mouth or nose (mouth-to-mouth resuscitation) or using a device that pushes air into the subject's lungs (mechanical ventilation).

### SUMMARY

The following embodiments and aspects thereof are described and illustrated in conjunction with systems, tools and methods which are meant to be exemplary and illustrative, not limiting in scope.

There is provided, in accordance with an embodiment, a resuscitation device facilitating the administration of cardiopulmonary resuscitation to a subject, the resuscitation device including a housing having a top surface and a bottom surface, the top surface having a concave dell configured to guide on the top surface a hand positioning of a rescuer administrating a cardiopulmonary resuscitation to a subject, and the bottom surface configured to position and stabilize the housing over a sternum of the subject, where the housing is configured to transmit a uniform distribution of the cardiopulmonary resuscitation force to the chest of the subject, the uniform distribution facilitates distributing the cardiopulmonary resuscitation force over a surface area that greater than the area of the top surface that directly receives the cardiopulmonary resuscitation force, thereby facilitating injury and contusion prevention to ribs and the sternum of the subject.

In some embodiments, the bottom surface includes a friction surface to prevent the housing from dislocating from its position on the chest of the subject during administration of the cardiopulmonary resuscitation force.

In some embodiments, the concave dell includes a secondary concave dell configured to facilitate administration of a cardiopulmonary resuscitation force to an infant.

In some embodiments, the resuscitation device further includes a compression indicator configured to provide an indication of a compression rhythm for application of the cardiopulmonary resuscitation force.

In some embodiments, the resuscitation device further includes a tarp operative to facilitate covering the chest of the subject during administration of a cardiopulmonary resuscitation force.

In some embodiments, the resuscitation device further includes at least one sensor configured to measure vital signs of the subject; and, at least one processing unit configured to monitor the vital signs measured by the at least one sensor, determine whether the housing is properly positioned on the chest according to the monitoring of the vital signs, generate a notification including instructions for administering the cardiopulmonary resuscitation force, providing in real-time, continuous feedback regarding the treatment administered and condition of the subject.

In some embodiments, the resuscitation device further includes a communication unit configured to communicate with a mobile device, the mobile device configured to present the user with user interface and instructional content.

In some embodiments, the resuscitation device further includes a second concave dell configured to facilitate administration of the cardiopulmonary resuscitation force during cardiopulmonary resuscitation of an infant.

In addition to the exemplary aspects and embodiments described above, further aspects and embodiments will become apparent by reference to the figures and by study of the following detailed description.

### BRIEF DESCRIPTION OF THE DRAWINGS

Some non-limiting exemplary embodiments or features of the disclosed subject matter are illustrated in the following drawings.

FIG. 1 shows a resuscitation device positioned on a chest of a subject to facilitate cardiopulmonary resuscitation, according to certain exemplary embodiments;

FIGS. 2A-2F show the resuscitation device of FIG. 1, according to certain exemplary embodiments;

FIG. 3 schematically illustrates the resuscitation device of FIGS. 1-2F, according to certain exemplary embodiments;

FIG. 4 outlines operations of a method executed by the resuscitation device of FIG. 1 to connect with a mobile device, according to certain embodiments; and,

FIG. 5 outlines operations of a method executed by the resuscitation device of FIG. 1 to assist in a treatment administer with cardiopulmonary resuscitation, according to exemplary embodiments.

Identical, duplicate, equivalent or similar structures, elements, or parts that appear in one or more drawings are generally labeled with the same reference numeral, optionally with an additional letter or letters to distinguish between similar entities or variants of entities, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

References to previously presented elements are implied without necessarily further citing the drawing or description in which they appear.

### DETAILED DESCRIPTION

Disclosed herein is a system and method for administering cardiopulmonary resuscitation (“CPR”), according to certain exemplary embodiments.

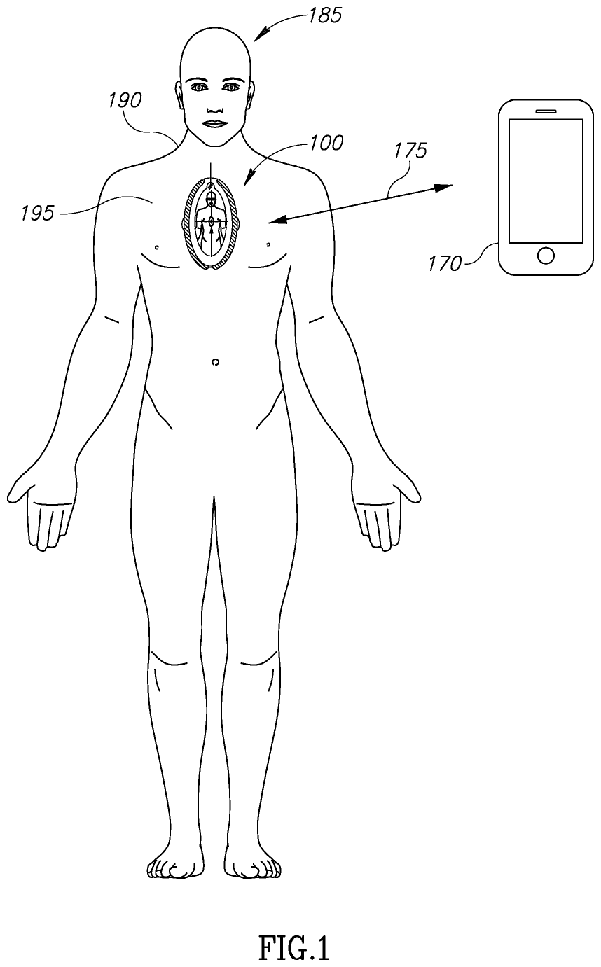

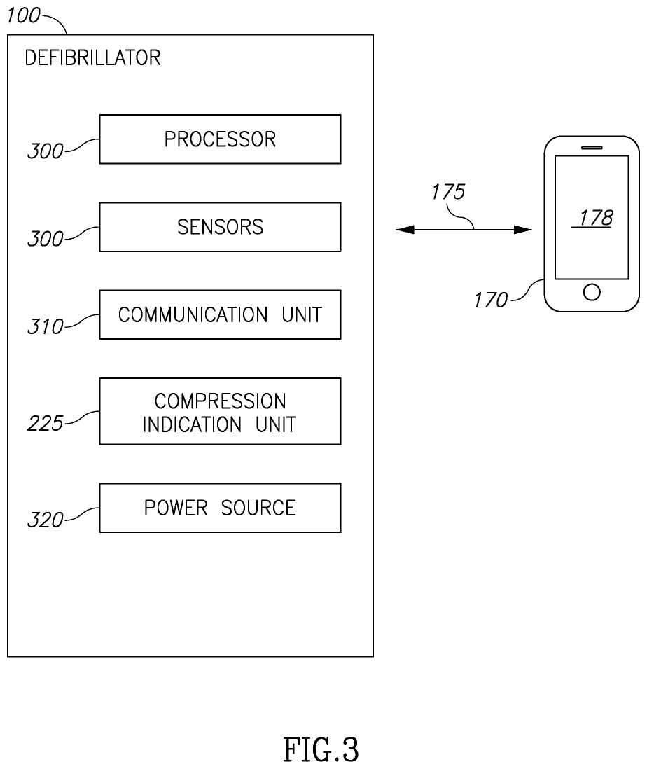

FIG. 1 schematically illustrates a resuscitation device 100 positioned on a chest 190 of a subject 185 for the administration of CPR, according to certain exemplary embodiments. Resuscitation device 100 is configured to be positioned on top of a sternum 195 subject 185 thereby dispersing a CPR force applied by a rescuer to ensure an effective administration of CPR. In some embodiments, resuscitation device 100 is configured to establish a connection, represented by arrow 175, with a mobile device 170 operated by the rescuer or an emergency medical technician (“EMT”) as described in conjunction with FIGS. 3-5. Through the connection, resuscitation device 100 can notify the EMT of the arrhythmia event and request medical assistance.

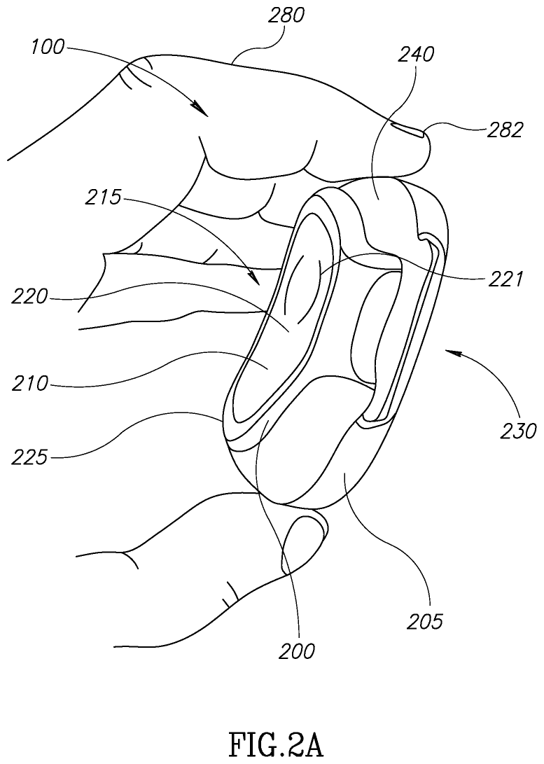

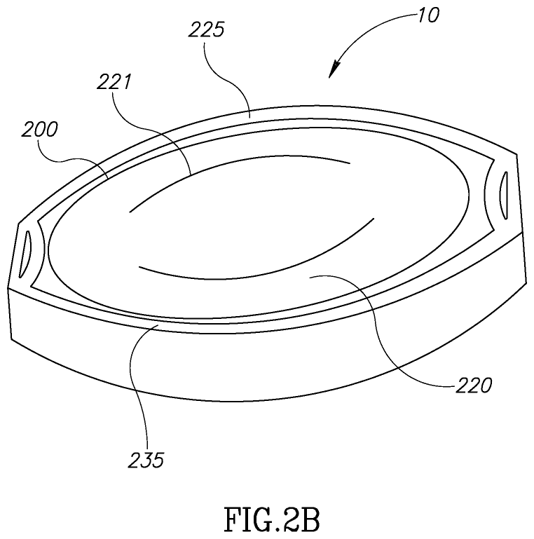

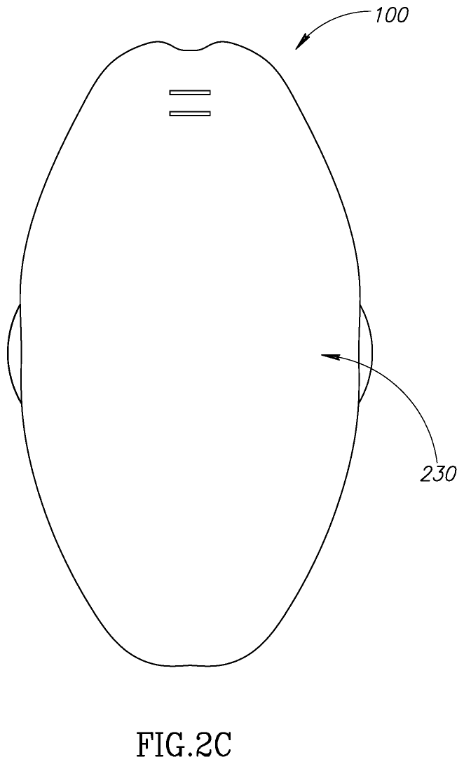

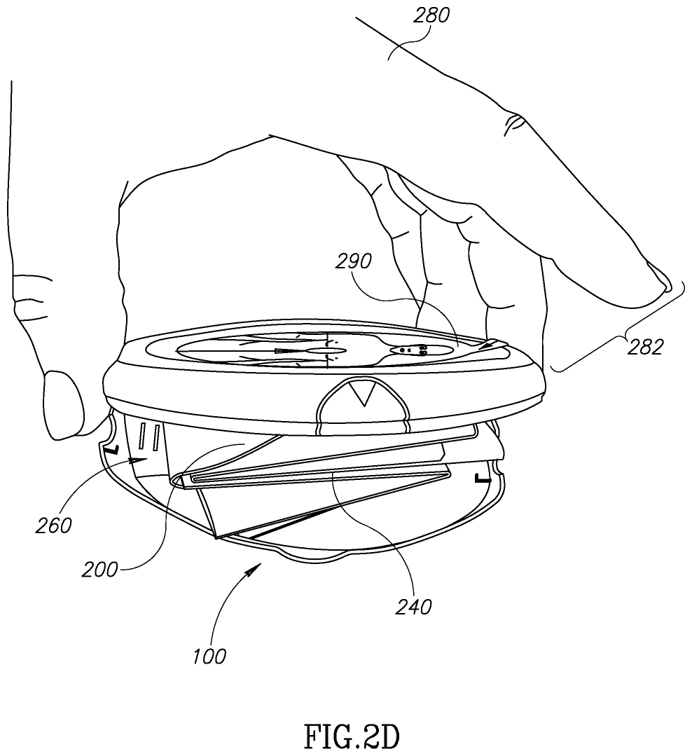

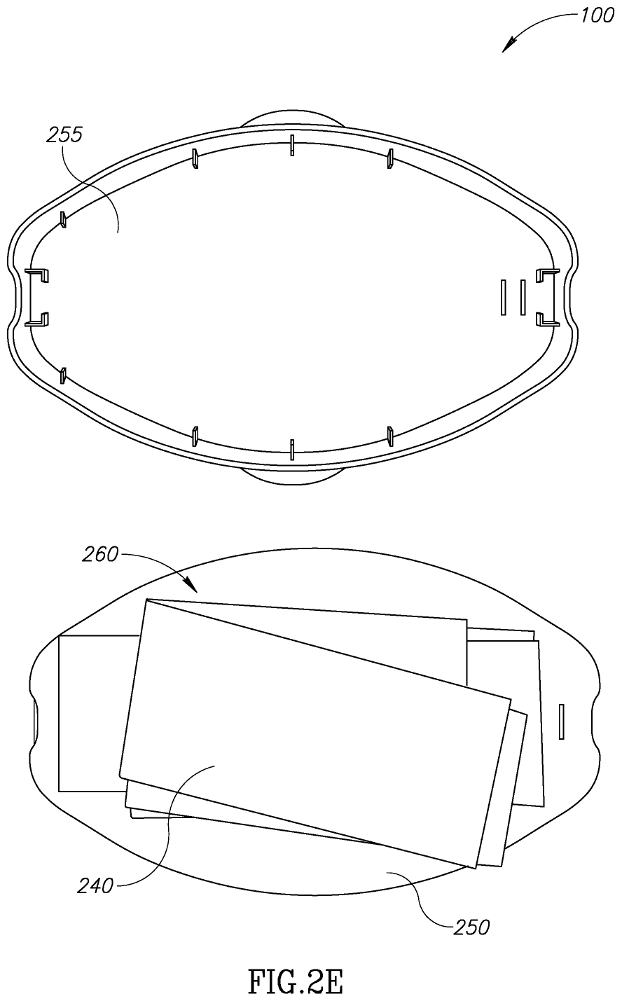

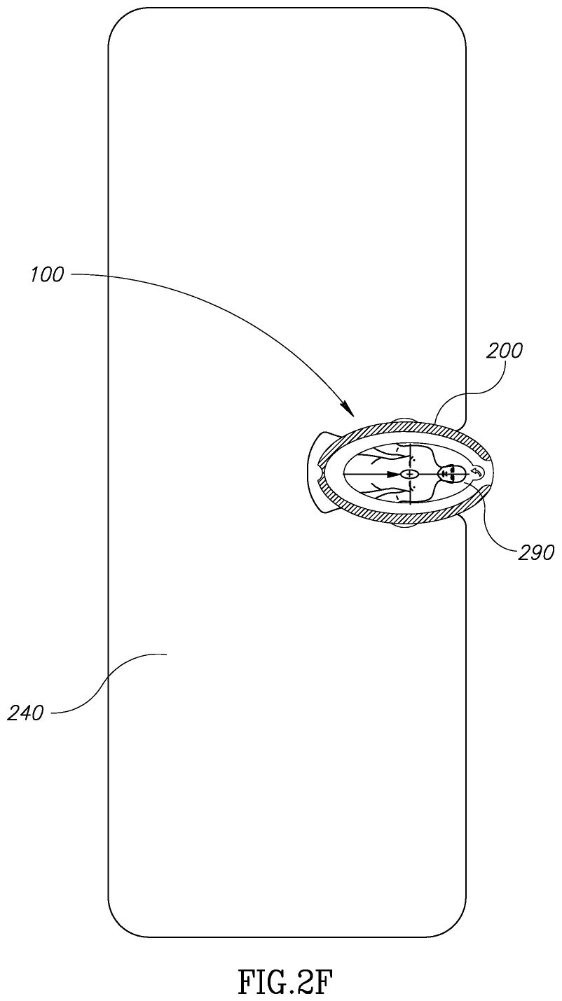

FIG. 2A-2C schematically illustrates resuscitation device 100, according to certain exemplary embodiments. FIG. 2A shows resuscitation device 100 having a housing 200 stored in a case 205 according to certain embodiments. Case 205 is configured to protect resuscitation device 100 when it is not in use. Housing 200 includes a central portion 210 on a top surface 215 of housing 200. Central portion 215 includes a concave dell 220 configured to facilitate proper positioning hands 280 of the rescuer to ensure a uniform distribution of CPR force during CPR. In certain embodiments, central portion 210 includes a secondary concave dell 221 configured to facilitate the administration of CPR to an infant. The secondary concave dell 221 is configured for proper positioning of one or more fingers 282 of the rescuer to facilitate uniform distribution of CPR force during the infant CPR. Housing 200 can include a compression indicator 225 which can be aligned along a border of central portion 210. Compression indicator 225 is configured to provide the rescuer with a rhythm at which to administer the CPR. In certain exemplary cases, compression indicator 225 can provide a visual indication, such as a flashing light, a sound indication, such as beeping noise, a motion indication, such as a vibration, or the like.

Referring to FIG. 2B, showing resuscitation device 100 extracted from case 205, according to certain exemplary embodiments.

Referring to FIG. 2C, showing a bottom surface 230 of resuscitation device 100, according to certain exemplary embodiments. Bottom surface 230 is configured for optimal positioning on sternum 195 (FIG. 1) thereby ensuring a uniform distribution of CPR force thereby ensuring uniform administration of CPR. Bottom surface 230 can include an adhesive layer (not shown) or having a compound that produces sufficient friction with chest 190 (FIG. 1) to prevent resuscitation device 100 from deviating from its positioning on top of sternum 195. For example, compound produces dry static friction between sternum 195 and resuscitation device 100 to prevent resuscitation device 100 from deviating from its positioning when a downward CPR force is applied to resuscitation device 100 by rescuer.

Referring to FIG. 2D, showing resuscitation device 100 during case 205 removal, according to certain embodiments. When resuscitation device 100 is removed from case 205, a tarp 240 can be extracted from a storage compartment 260 that is housed between a top portion 255 and a bottom portion 250 of resuscitation device 100. After resuscitation device 100 is removed from case 205, a bottom surface 230 of housing 200, is exposed and can be positioned on sternum 195 (FIG. 1). In some embodiments, resuscitation device 100 includes an instructional cover or sticker, referenced as 290, which illustrates for the rescuer the proper location for positioning resuscitation device 100 on subject 185. The instruction cover 290 can be removed to prevent it from interfering with CPR.

Referring to FIG. 2E, showing resuscitation device 100 having top portion 255 and bottom portion 250 separated to expose a storage compartment 260, according to certain exemplary embodiments. Storage compartment 260 stores tarp 240, which is folded to fit within storage compartment 260.

Referring to FIG. 2F, once removed from case 205, tarp 240 is extracted and expanded to cover chest of subject 190 (FIG. 1) during CPR, according to certain exemplary embodiments. In some embodiments, tarp 240 covers chest 190 of a female subject thereby alleviating any discomfort that can arise from the administration of medical assistance that requires removal of the clothing from subject 185 (FIG. 1). In some embodiments, tarp 240 can be utilized as personal protective equipment (“PPE”) to prevent contact between subject 185 and the rescuer. Due to the close interaction between the subject 185 and rescuer during CPR, there is a high likelihood of physical contact between them and which increases the likelihood of transmission of viruses, bacteria, or the like resulting in infection, disease, or the like. Tarp 240 provides sufficient surface coverage to reduce potential contact area between the subject and the rescuer.

FIG. 3 schematically illustrates resuscitation device 100, according to certain embodiments. Resuscitation device includes a power source 320, one or more sensors 305, a communication unit 310, an indication unit 315 and a processor 300. Sensors 305 are configured to measure the vital signs of subject 185 (FIG. 1) when there is contact between resuscitation device 100 and chest 190 (FIG. 1). The vital signs can include heart rate, blood pressure, breathing rate, or the like, as well as information regarding the compression rate and depth. Sensors 305 are embedded within bottom surface 230 thereby enabling sensors 305 to be in physical contact with chest 190. In certain embodiments, sensors 305 can include an accelerometer configured to measure a depth and rate of the compressions administered during CPR and can include an acoustic sonogram configured to measure a cardiac filling and output.

Communication unit 310 is configured to connect and communicate, represented by arrow 175, with mobile device 170 of an EMT to notify of the emergency and request assistance. In some embodiments, communication unit 310 is configured to connect and communicate 175 with mobile device 170 of the rescuer, thereby activating an instructional application that demonstrates for the rescuer the proper hand positioning on resuscitation device 100. For example, the instructions application displays an instructional video along with audio instructions.

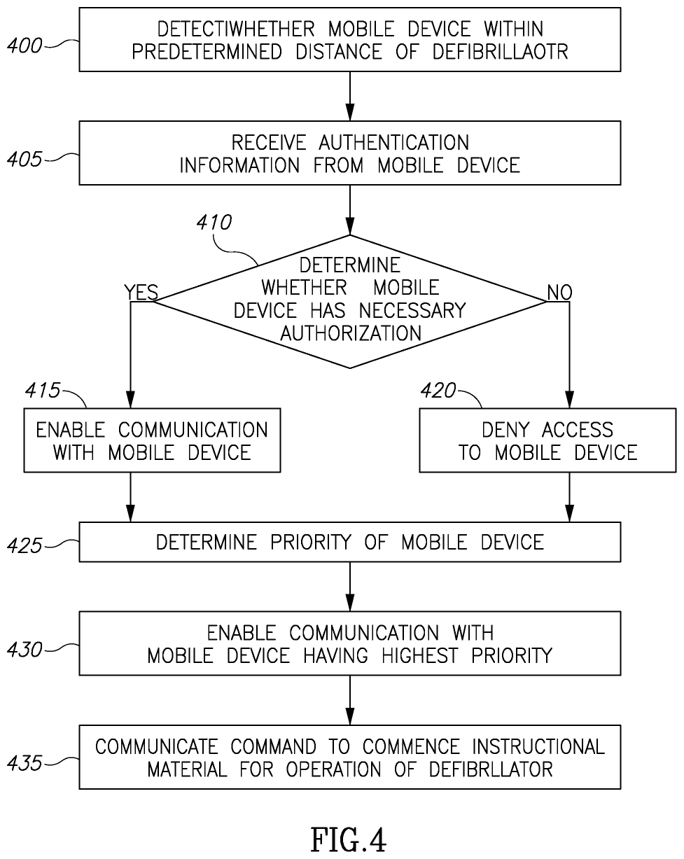

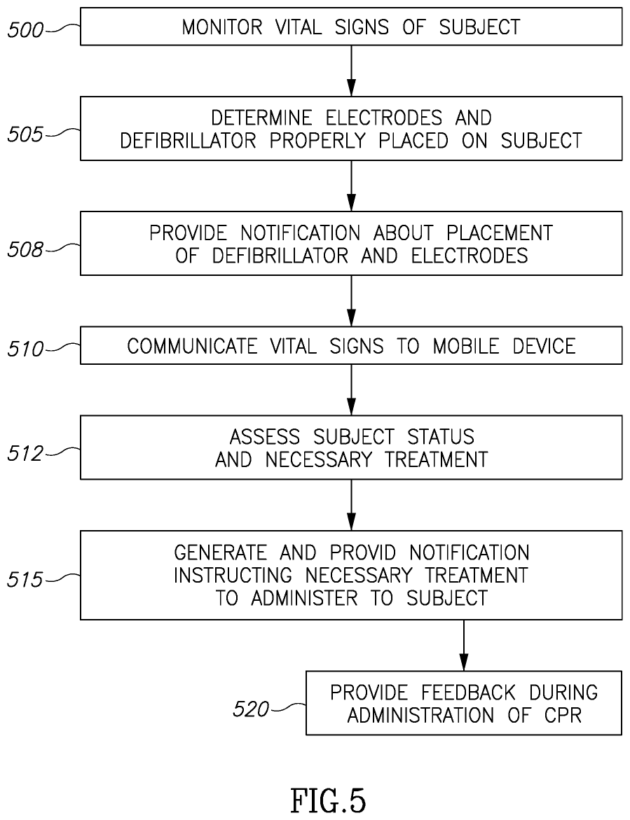

Compression indication unit 225 is configured to provide a visual, audio and/or motion assistive indication to the rescuer of the necessary compression rhythm and depth. In some embodiments, the indication provided can be customized according to subject 185 (FIG. 1), for example, according to the age, gender, or the like of subject 185. Processor 300 is configured to execute operations described in conjunction with FIGS. 4-5.

In some optional embodiments, compression indication unit 225 can provide an indication that bottom surface 230 is not positioned on top of sternum 195 (FIG. 1). The indication can be according to a determination made by processor 300 that sensors 305 are not measuring the vital signs and the processor 300 operates compression indication unit 225 to generate an indication that the resuscitation device 100 has to be repositioned on top of sternum 195.

Mobile device 170 can execute an application that provides a user of mobile device 170, such as a rescuer, with a user interface 330 that enables the rescuer to communicate with resuscitation device 100 and monitor the vital signs and condition of subject 190. User interface 330 can present video and audio instructions to the rescuer facilitating the operation of resuscitation device 100. User interface 330 can display an instructional video presentation providing step-by-step instructions to the user to correctly operate resuscitation device 100, for example, a video and audio instructional that after each instructional step gives the provider time to perform the step. Resuscitation device 100 can provide mobile device 170 with real-time feedback about performance of the step and user interface 330 can provide the rescuer with additional instructional material, repeat the instructional step or continue to the next step according to the performance of the rescuer.

Reference is now made to FIG. 4, outlining operations of a method executed by processor 300 (FIG. 3) to associate resuscitation device 100 (FIG. 3) with mobile device 170 (FIG. 3), according to certain embodiments. When emergency services are notified of a medical emergency requiring operation of resuscitation device 100, the emergency services can notify a rescuer of the incident and the rescuer is dispatched to the event. The rescuer can be dispatched according to vicinity to the resuscitation device 100, which can be determined by registration of resuscitation device 100 with a crowdsourcing platform or with medical services within a predetermined area, such as a city, district, state, and/or the like.

When the rescuer is within a predetermined distance from resuscitation device 100, processor 300 executes operation 400 detecting whether mobile device 170 is within a predetermined distance from resuscitation device 100. Processor 300 detects whether mobile device 170 is within a predetermined distance from resuscitation device 100, for example from pings received from mobile devices near resuscitation device 100. For example, the distance can be within a 1-meter radius from a location of resuscitation device 100.

In operation 405, processor 300 receives authentication information from mobile device 170. Communication unit 310 (FIG. 3) received the authentication information from mobile device 170, which is provided to processor 300.

In operation 410, processor 300 determines whether mobile device 170 has a necessary authorization to enable communication between to resuscitation device 100 and mobile device 170. Processor 300 compares the authentication information received from mobile device 170 with stored authentication information. The stored authentication information can be authentication information that is registered with resuscitation device 100 prior to a medical emergency, for example, during production, first time activation and/or the like. The stored authentication information can include authentication information for all rescuers within a predetermined area such as a city, district, and/or the like, or can be of predetermined organizations such as the red cross, first aid responders and/or the like. In certain embodiments, registration can be achieved through crowdsourcing platforms.

Where the authentication information matches the stored authentication information, processor 300 executes operation 415 enabling communication 175 (FIG. 3) between resuscitation device 100 and mobile device 170. Where the authentication information does not match the stored authentication information mobile device 170, processor 300 executes operation 420 denying mobile device 170 access to resuscitation device.

In certain exemplary embodiments, multiple mobile devices may have authentication information that will enable communication with resuscitation device 100. In such cases a hierarchy of authentication can be stored in resuscitation device 100, and processor 300 executes optional operation 425 to determine a priority of mobile device 170. According to the priority of mobile device 170, processor 300 prioritizes mobile device 170 over other mobile devices according to the hierarchy of authentication based on the authentication information of mobile device 170 and executes operation 430 to enable communication with mobile device 170 with highest priority.

In operation 435, processor 300 provides a command to commence instructional material of operating resuscitation device 100. Processor 300 provides communication unit 310 with a command that is transferred to mobile device 170 to begin an instructional presentation in the application of mobile device 170. The instructional presentation can be in the form of a video and audio presentation that shows the user of mobile device 170 how to place resuscitation device 100, how to place hands for CPR and how to administer CPR.

FIG. 5 outlines operations of a method executed by processor 300 (FIG. 3) to assist CPR administration to subject 185 (FIG. 1), according to exemplary embodiments.

In operation 500, processor 300 monitors vital signs. Processor 155 operates sensors 160 (FIG. 3) to monitor and collect data associated with vital signs of subject 185 as described in conjunction with FIG. 3.

In operation 505, processor 300 determines whether resuscitation device 100 has been properly positioned on chest 190 (FIG. 1). Processor 300 determines the proper position according to the vital signs measured by sensors 160 (FIG. 3). Sensors 160 embedded in bottom surface 230 measure the vital signs when bottom surface 230 is in contact with chest 190 at the predetermined optimal position for administration of CPR through which processor 300 can determine that bottom surface 230 is positioned in the correct location on top of sternum 195 (FIG. 1).

In operation 508, processor 300 transmits a notification to mobile device 170 about the positioning of resuscitation device 100. The notification is provided to mobile device 170, which can be viewed via user interface 178 (FIG. 1). The notification can inform that the resuscitation device 100 is properly positioned on chest 190, or that resuscitation device 100 is not correctly positioned and must be moved by a rescuer, after which processor 155 performs steps 500, 505 and 508 until resuscitation device 100 and electrodes 120, 125 are properly placed.

In operation 510 processor 300 operates communication unit 310 to communicate the vital signs to mobile device 170, which can then be displayed on user interface 330 (FIG. 3).

In operation 512, processor 300 assesses the status of subject 190 and the necessary treatment. Processor 300 analyzes the vital signs and determines the treatment necessary to subject 190. For example, processor 300 can determine subject 190 is suffering from heart arrythmia and therefore an electric pulse must be administered to subject 190, or that CPR must be administered to subject 190.

In operation 520, processor 300 provides feedback and instruction for proper administration of CPR by the rescuer.

In the context of some embodiments of the present disclosure, by way of example and without limiting, terms such as ‘operating’ or ‘executing’ also imply capabilities, such as ‘operable’ or ‘executable’, respectively.

Conjugated terms such as, by way of example, ‘a thing property’ implies a property of the thing, unless otherwise clearly evident from the context thereof.

The terms ‘processor’ or ‘computer’, or system thereof, are used herein as ordinary context of the art, such as a general purpose processor or a micro-processor, RISC processor, or DSP, possibly comprising additional elements such as memory or communication ports. Optionally or additionally, the terms ‘processor’ or ‘computer’ or derivatives thereof denote an apparatus that is capable of carrying out a provided or an incorporated program and/or is capable of controlling and/or accessing data storage apparatus and/or other apparatus such as input and output ports. The terms ‘processor’ or ‘computer’ denote also a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘code’ or ‘application’ may be used interchangeably according to the context thereof, and denote one or more instructions or directives or circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry.

The processor and program may constitute the same apparatus, at least partially, such as an array of electronic gates, such as FPGA or ASIC, designed to perform a programmed sequence of operations, optionally comprising or linked with a processor or other circuitry.

The term computerized apparatus or a computerized system or a similar term denotes an apparatus comprising one or more processors operable or operating according to one or more programs.

As used herein, without limiting, a module represents a part of a system, such as a part of a program operating or interacting with one or more other parts on the same unit or on a different unit, or an electronic component or assembly for interacting with one or more other components.

As used herein, without limiting, a process represents a collection of operations for achieving a certain objective or an outcome.

As used herein, the term ‘server’ denotes a computerized apparatus providing data and/or operational service or services to one or more other apparatuses.

The term ‘configuring’ and/or ‘adapting’ for an objective, or a variation thereof, implies using at least a software and/or electronic circuit and/or auxiliary apparatus designed and/or implemented and/or operable or operative to achieve the objective.

A device storing and/or comprising a program and/or data constitutes an article of manufacture. Unless otherwise specified, the program and/or data are stored in or on a non-transitory medium.

In case electrical or electronic equipment is disclosed it is assumed that an appropriate power supply is used for the operation thereof.

The flowchart and block diagrams illustrate architecture, functionality or an operation of possible implementations of systems, methods and computer program products according to various embodiments of the present disclosed subject matter. In this regard, each block in the flowchart or block diagrams may represent a module, segment, or portion of program code, which includes one or more executable instructions for implementing the specified logical function(s). It should also be noted that, in some alternative implementations, illustrated or described operations may occur in a different order or in combination or as concurrent operations instead of sequential operations to achieve the same or equivalent effect.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

As used herein the term “configuring” and/or ‘adapting’ for an objective, or a variation thereof, implies using materials and/or components in a manner designed for and/or implemented and/or operable or operative to achieve the objective.

Unless otherwise specified, the terms ‘about’ and/or ‘close’ with respect to a magnitude or a numerical value implies within an inclusive range of −10% to +10% of the respective magnitude or value.

Unless otherwise specified, the terms ‘about’ and/or ‘close’ with respect to a dimension or extent, such as length, implies within an inclusive range of −10% to +10% of the respective dimension or extent.

Unless otherwise specified, the terms ‘about’ or ‘close’ imply at or in a region of, or close to a location or a part of an object relative to other parts or regions of the object.

When a range of values is recited, it is merely for convenience or brevity and includes all the possible sub-ranges as well as individual numerical values within and about the boundary of that range. Any numeric value, unless otherwise specified, includes also practical close values enabling an embodiment or a method, and integral values do not exclude fractional values. A sub-range values and practical close values should be considered as specifically disclosed values. As used herein, ellipsis ( . . . ) between two entities or values denotes an inclusive range of entities or values, respectively. For example, A . . . Z implies all the letters from A to Z, inclusively.

The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded. Terms in the claims that follow should be interpreted, without limiting, as characterized or described in the specification.

1. A resuscitation device facilitating the administration of cardiopulmonary resuscitation to a subject, the resuscitation device comprising:
a housing having a top surface and a bottom surface, said top surface having a concave dell configured to guide on the top surface a hand positioning of a rescuer administrating a cardiopulmonary resuscitation to a subject, and said bottom surface configured to position and stabilize the housing over a sternum of the subject; and,
wherein the housing is configured to transmit a uniform distribution of the cardiopulmonary resuscitation force to the chest of the subject, said uniform distribution facilitates distributing the cardiopulmonary resuscitation force over a surface area that greater than the area of the top surface that directly receives the cardiopulmonary resuscitation force, thereby facilitating injury and contusion prevention to ribs and the sternum of the subject.

2. A resuscitation device according to claim 1, wherein said bottom surface comprises a friction surface to prevent the housing from dislocating from its position on the chest of the subject during administration of the cardiopulmonary resuscitation force.

3. A resuscitation device according to claim 1, wherein said concave dell comprises a secondary concave dell configured to facilitate administration of a cardiopulmonary resuscitation force to an infant.

4. A resuscitation device according to claim 1, further comprising a compression indicator configured to provide an indication of a compression rhythm for application of the cardiopulmonary resuscitation force.

5. A resuscitation device according to claim 1, further comprising a tarp operative to facilitate covering the chest of the subject during administration of a cardiopulmonary resuscitation force.

6. A resuscitation device according to claim 1, further comprising:
at least one sensor configured to measure vital signs of the subject; and,
at least one processing unit configured to:
monitor the vital signs measured by the at least one sensor;
determine whether the housing is properly positioned on the chest according to the monitoring of the vital signs;
generate a notification comprising instructions for administering the cardiopulmonary resuscitation force; and,
providing real-time, continuous feedback regarding the treatment administered and condition of the subject.

7. A resuscitation device according to claim 1, further comprising a communication unit configured to communicate with a mobile device, the mobile device configured to present the user with user interface and instructional content.

8. A resuscitation device according to claim 1, further comprising a second concave dell configured to facilitate administration of the cardiopulmonary resuscitation force during cardiopulmonary resuscitation of an infant.
